(19) United States
(12) Patent Application Publication
(10) Pub. No.: US 2026/0069748 A1
(43) Pub. Date: Mar. 12, 2026

(54) COATING MATERIALS AND METHODS FOR MEDICAL DEVICES

(71) Applicant: Piezaolabs Medical Inc., Lake Forest, CA (US)

(72) Inventor: Jianlu Ma, Irvine, CA (US)

(73) Assignee: Piezolabs Medical Inc., Lake Forest, CA (US)

(21) Appl. No.: 18/883,941

(22) Filed: Sep. 12, 2024

(51) Int. Cl. A61L 31/10

(52) U.S. Cl. CPC A61L 31/10; A61L 2300/608

(57) ABSTRACT

An implantable medical device has a structural framework having a base material that is coated with a surface enhancement layer. The coated structural framework is then poled with a desired polarity that carries either a positive charge or a negative charge on the surface of the coated structural framework when the structural framework is under deformation. The surface enhancement layer can be a copolymer or a zinc oxide coating, or can include some portions that are the copolymer and some portions that are the zinc oxide coating.

### BACKGROUND OF THE INVENTION

### 1. Field of the Invention

The present invention relates to medical devices that are made from either biocompatible metallic material or biocompatible polymer materials, and at least partially or completely coated with an enhancement surface layer for improved device functionality.

### 2. Description of the Prior Art

There are many vascular devices on the market, but there are still many unmet clinical needs. For example, for the devices used in the main vascular systems, such as stents implanted in the arteries or veins to maintain the patency of the vessels, heart valves, and similar implants in the circulation systems, dual antiplatelet therapy (DAPT) use following the intervention procedure is still required. The ideal situation is to shorten or eliminate the DAPT duration or to change to a single APT therapy. For devices that are used to seal or occlude vascular abnormities, such as aneurysms, AVMs, cavernous malformations, ruptured vessels, endoleaks, and similar abnormalities, incomplete occlusion and delayed occlusion/healing are still major issues that need to be addressed.

In summary, an improved functionality/healing effect is still needed for such implantable devices that avoids the drawbacks mentioned above.

### SUMMARY OF THE DISCLOSURE

The present invention seeks to provide new coating materials and/or techniques to address the unmet clinical needs mentioned hereinabove.

In particular, the present invention provides a surface enhancement layer that contains materials/composition exhibiting piezoelectric properties.

To meet the objectives of the present invention, there is provided an implantable medical device that has a structural framework having a base material that is coated with a surface enhancement layer. The coated structural framework is then poled with a desired polarity that carries either a positive charge or a negative charge on the blood contact surface of the device.

In one embodiment, the surface enhancement layer is a polymeric coating comprising a copolymer polymerized onto the surface of the structural framework from at least one monomer unit of (—CF2-CFH—) and at least one monomer unit of —CH2-CF2-, wherein at least one monomer has a Glass Transition Temperature (Tg) less than about 95 degrees Celsius, and wherein in homopolymer form, the at least one monomer unit of (—CF2-CFH—) in the copolymer is protonated.

In another embodiment, the surface enhancement layer is zinc oxide.

In another embodiment, the surface enhancement layer can include some portions that are the copolymer and some portions that are the zinc oxide coating.

In one embodiment, the medical device is a vascular stent, such that once implanted, under deformation, the blood contact surface (usually the inner surface) of the vascular stents generates negative surface charge, while the outer surface of the vascular stent generates positive surface charge.

In another embodiment, the medical device is a vascular occlusion device, such as an embolic coil or a vascular plug, such that once implanted, under deformation, the blood contact surface (usually the outer surface) of the vascular occlusion device generates positive surface charge, while the inner side of the vascular occlusion device generates negative surface charge.

Thus, the surface enhancement layer comprises at least one material having piezoelectric property. The surface enhancement layer alters the interaction between the surface of the medical device and body fluid, including blood.

### BRIEF DESCRIPTION OF THE DRAWINGS

FIG. 1 is a schematic side view of a braided stent according to one embodiment of the present invention shown without the surface enhancement layer.

FIG. 2 is a perspective view of the stent of FIG. 1.

FIG. 3 is an end view of the stent of FIG. 1.

FIG. 4 is a schematic side view of the stent of FIG. 1 with the surface enhancement layer provided on the inner surface of the stent.

FIG. 5 is a perspective view of the stent of FIG. 4.

FIG. 6 is an end view of the stent of FIG. 4.

FIG. 7 is a schematic side view of a stent structure with the surface enhancement layer provided on the outer surface of the stent.

FIG. 8 is an end view of the stent of FIG. 7.

FIG. 9 is a schematic side view of a stent structure with the surface enhancement layer provided only in the cell space between the wires of the stent

FIG. 10 is a schematic side view of the stent of FIG. 1 with the surface enhancement layer applied only on the wire of the stent.

FIG. 11A is an end view of the stent of FIG. 10.

FIG. 11B is a cut-away sectional view of a portion of the stent of FIG. 10 showing the surface enhancement layer over the wires.

FIG. 12 is a perspective view of a stent showing a modification made to the stent of FIG. 7.

FIGS. 13A-13C illustrate the alignment of electric dipoles represented by arrows in a piezoelectric material prior to poling, during the poling process, and at the end of poling, respectively.

FIGS. 14a-14d illustrate the piezoelectric effect where mechanical tension or compression on a piezoelectric element will induce a surface charge with the polarity depending on the type of stress and direction.

FIG. 15 illustrates the poling apparatus for poling the stents of FIGS. 4-12.

FIG. 16 is a schematic side view of a stent according to another embodiment of the present invention.

FIG. 17 illustrates a three-dimensional embolic coil according to the present invention that that has been delivered to an aneurysm sac by a delivery device.

FIG. 18 is a schematic view of a two-dimensional embolic coil according to the present invention.

FIG. 19 is a schematic view of the three-dimensional embolic coil of FIG. 17.

FIG. 20 illustrates an apparatus for poling the embolic coil in FIG. 18.

FIG. 21 illustrates another apparatus for poling the embolic coil in FIG. 18.

FIG. 22 illustrates an apparatus for poling the embolic coil in FIG. 19.

FIGS. 23A and 23B illustrate the test results for a stent that is made according to one embodiment of the present invention.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a surface enhancement layer that is applied to the surface of an implantable medical device. The surface enhancement layer contains materials/composition exhibiting piezoelectric properties.

The medical device is at least partially made of a biocompatible metallic or polymer material, and has a biologically compatible surface enhancement layer. The medical device is a vascular implantable device, and can include, but is not limited to, stents (e.g., stents for used in all parts of the human vasculature-blood circulation system, body lumens, etc), vascular/aneurysm occlusion devices (e.g., devices to occlude aneurysms or vessels in the blood circulation system, other bodily lumens, vasculature, etc.), vascular implants, heart valves, and similar devices. The medical device can include an expandable body, such as a stent. For example, the medical device can include a braided structure with an expandable body to maintain the patency of the main vessel, and/or to occlude the aneurysm, or other abnormalities in the vasculature or other bodily lumen. In another non-limiting embodiment, the medical device can be a prosthetic heart valve with an expandable frame. In another non-limiting embodiment, the medical device can be a prosthetic heart valve with moveable leaflets, and the surface enhancement layer can be partially or fully coated onto the moveable leaflets.

The metallic or polymer material that is used to partially or fully form the medical device is optionally subject to one or more manufacturing processes. These manufacturing processes can include, but are not limited to, laser cutting, sand basting, acid cleaning, chemical etching, expansion, shape setting, electropolishing, electroplating, heat treatment, machining, and plasma coating, 3D printing, 3D printed coating, chemical vapor deposition (CVD), plasma enhanced CVD (PE-CVD), dip coating, spray coating, ultrasound spray coating, ion beam deposition, sputtering coating, vacuum deposition, physical vapor deposition (PVD), hot dip galvanized zinc (HDG) coating, coating by electroplating, atomic layer deposition (ALD), etc.

The biocompatible materials that can be used for medical device of the present invention include biocompatible metallic materials and polymer materials.

The metallic materials can include stainless Steel (e.g., 316L); Cobalt-Chromium alloys (e.g., L605, MP35N, etc.); Nickel-Titanium alloys (e.g., Nitinol, Ni-rich Nitinol, Ti-Rich nitinol, etc.) and their ternary alloys or quaternary; Titanium and its alloys; Platinum and its alloys; Tantalum and it alloys; Molybdenum and it alloys; Platinum (Pt) and its alloys, and Tungsten (W) and its alloys; among others.

The polymer materials can include Polystyrene (PS), PTFE, PVDF, PVDF and it's copolymers, P(VDF-TrFE), Polypropylene (PP), Polyvinyl chloride (PVC), Polyethylene (PE), Polyurethane (PU), Polycarbonate (PC), Polyethylene terephthalate (PET), Polyetheretherketone (PEEK), parylene C, D, PLA, PLLA, and PHB, among others. This can include the co-polymers, composite polymers, and combination of two or more different polymers listed above. The polymer materials can either be biodegradable, bio-absorbable, or non-degradable or non-absorbable.

The surface enhancement layer can either partially or completely cover the entire surface area of the medical device. The surface enhancement layer contains materials/composition exhibiting piezoelectric property. Once implanted, under the physiological conditions, such as vessel wall movement, body fluid interaction, blood interaction, blood flow, etc., the surface enhancement layer can self-generate surface charge, and the surface charge alters/controls the device-blood interaction. Since most of the blood components (red blood cell, proteins, platelets, etc.) carry a negative surface charge, the device-blood interaction can be controlled by the polarity of the surface enhancement layer under physiological conditions. For example, under the physiological conditions, if the blood contact surface on the medical device generates a negative charge, then the medical device's surface can expel blood components, thereby preventing thrombosis, stenosis and restenosis, to keep the blood vessel open. In contrast, under the physiological conditions, if the blood contact surface on the medical device generates a positive charge, then the medical device's surface can attract blood components, thereby promoting thrombosis, so as to speed up the occlusion process. The polarity of the device under physiological conditions can be controlled/engineered during the manufacturing process for the medical device.

The surface enhancement layer that exhibits piezoelectric property disclosed in the present invention is primarily a copolymer or a ZnO coating. However, more generally, the surface enhancement layer can include poly(vinylidene fluoride) (PVDF), P(VDF-co-trifluoroethylene) (P(VDF-TrFE)) Copolymers, P(VDF-TRFE-CTFE), P(VDF-TrFE-CFE) Terpolymers, polysaccharides (cellulose, chitin, and amylose), proteins (collagen, keratin, and fibrin), DNA, bio-polyesters (PLLA and PHAs), PLA, Nylon-11, Nylon-7 PEEK, ZnO, BaTiO3, PZT, Gallium nitride (GaN), MoS2, WSe2, TaO, Ag—ZnO, ZrO2, and other non-lead known piezoelectric materials.

Copolymer: One polymer material that exhibits the piezoelectric property is based on the poly(trifluoroethylene) (PTrFE) polymer. It can be synthesized with other polymers to create copolymers that have piezoelectric property after the proper annealing process and/or poling process. When poly(trifluoroethylene) (PTrFE) is synthesized with poly(vinylidene fluoride) (PVDF) polymer, the resulting co-polymer is P(VDF-TrFE). The chemical formula of P(VDF-TrFE) copolymer is shown below.

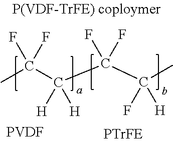

PVDF is a crystalline polymer, and has a monomer unit of —CH2-CF2-, in between polyethylene (PE) (—CH2-CH2-) and polytetrafluoroethylene (PTFE) (—CF2-CF2-) monomers. The similarity of PVDF to these two polymers gives rise to its physical strength, flexibility and chemical stability. Its ferroelectric properties originate from the large difference in electronegativity between fluorine, carbon and hydrogen, which have Pauling's values of 4.0, 2.5 and 2.1, respectively. Most of the electrons are attracted to the fluorine side of the polymer chain and polarization is created. The Curie temperature (the temperature at which certain materials undergo a change in their magnetic properties) of PVDF is estimated to be above the melting temperature at 195-197 degrees C. The melting of the ferroelectric phase and recrystallization to the paraelectric phase may happen in the same temperature range. The addition of TrFE (—CF2-CFH—) into the PVDF system plays an important role in the phase transition behavior. TrFE modifies the PVDF crystal structure by increasing the unit cell size and inter-planar distance of the ferroelectric phase. The interactions between each unit and between dipole-to-dipole are reduced, resulting in a lower Curie temperature. Therefore, it allows the copolymer to crystallize into the ferroelectric phase at temperatures below the melting point (170-190 degree C.). The copolymer crystal structure, phase transition behavior and ferroelectric properties are affected by the ratio of VDF/TrFE content and the synthesizing conditions. The ratio (by mol %) of VDF/TrFE can be 1/99, 5/95, 10/90, 15/85, 20/80, 25/75, 30/70, 35/65, 40/60, 45/55, 55/55, 60/40, 65/45, 70/30, 75/25, 80/20, 85/15, 90/10, 95/5, 99/1, etc. The Glass transition temperature (Tg) (i.e., the temperature at which molecular mobility begins to take place, below which molecular mobility is frozen and the elastomer becomes rigid and glassy) of the co-polymer is below −35° C. Suspension polymerization process is one of the many synthesization processes that can be used to synthesis the co-polymers.

The thickness of the surface enhancement layer is preferably in the range between 1 nanometers to 400 micrometers.

The surface charge generated on the surface of the medical device from the surface enhancement layer can reduce bacterial growth and bacterial infection around the medical device.

The surface charge generated on the surface of the medical device from the surface enhancement layer can also suppress the inflammation reaction post stent implantation procedure, thereby suppressing in-stent restenosis post stent implantation.

The surface charge generated on the surface of the medical device from the surface enhancement layer can promote endothelial cell angiogenesis, thereby creating an improved pro-healing environment around the medical device.

The surface enhancement layer resists corrosion or ionization of the medical device, preventing the release of the ions that can lead to allergic reaction, such as Ni, and Cr ions. Resisting corrosion can also help to maintain the structural integrity of the medical device.

### The Mechanism of Poling

The background and mechanism of poling will be described in this section. In a macroscopic crystalline structure that comprises several such unit cells, the dipoles are by default found to be randomly oriented. When the material is subjected to a mechanical stress, each dipole rotates from its original orientation toward a direction that minimizes the overall electrical and mechanical energy stored in the dipole. If all the dipoles are initially randomly oriented (i.e. a net polarization of zero), their rotation may not significantly change the macroscopic net polarization of the material, hence the piezoelectric effect exhibited will be negligible. Therefore, it is important to create an initial state in the material such that most dipoles will be more-or-less oriented in the same direction. Such an initial state can be imparted to the material by poling it. The direction along which the dipoles align is known as the poling direction.

FIGS. 13A-13C illustrate the alignment of electric dipoles represented by arrows in a material prior to poling (FIG. 13A), during the poling process (FIG. 13B) and at the end of poling (FIG. 13C). The process of poling involves aligning all of these individual dipole moments, so that they all point in the same general direction. This is accomplished by putting the piezoelectric material in a constant electric field to force the dipoles to align. In the electric field each dipole will feel a torque if it is not parallel to the field lines produced, and so is turned to that direction. When the electric field is removed, the dipoles remain fairly aligned, although there will still be some element of random direction.

During poling, the material is subjected to a very high electric field that orients all the dipoles in the direction of the field. Upon switching off the electric field, most dipoles do not return back to their original orientation as a result of the pinning effect produced by microscopic defects in the crystalline lattice. This provides a material comprising numerous microscopic dipoles that are roughly oriented in the same direction. It is noteworthy that the material can be de-poled if it is subjected to a very high electric field oriented opposite to the poling direction or is exposed to a temperature higher than the Curie temperature of the piezoelectric material.

When a stress is applied to a piezoelectric material, the dimensions of the material changes. Depending on the direction the stress is applied, the resulting change in dimensions can shift the centers of mass for the positive and negative ions; this produces a dipole throughout the material. The dipoles inside the material cancel each other out, but on the surface of the material the dipoles are not canceled out, producing a polarity given by:

$$P = d T = d ( F / A )$$

where T is the mechanical stress, P is the induced polarization, and d is the piezoelectric coefficient.

Polarization charge is given by

$$P = Q / A$$

where
Q is the charge and
A is the cross-sectional area of the piezoelectric material the stress is applied on.

The polarization induces a voltage due to the given by

$$V = \frac{Q}{C} = \frac{AP}{\frac{{ϵ}_{0} {ϵ}_{r} A}{L}} = \frac{LP}{{ϵ}_{0} {ϵ}_{r}} = \frac{L ( d \frac{F}{A} )}{{ϵ}_{0} {ϵ}_{r}} = \frac{dLF}{{ϵ}_{0} {ϵ}_{r} A}$$

where
ϵ0 is the vacuum permittivity,
ϵr is the relative permittivity,
L is the length of the piezoelectric material, and
C is the capacitance.

On the other hand, if an electric field is applied to the piezoelectric material, it produces a strain given by

$$S = d E$$

where E is the applied electric field, S is the produced strain, and d is the same piezoelectric coefficient as above. This is also known as the converse piezoelectric effect. In this case, negative ions would tend to move towards the positive end of the electric field, and the positive ions towards the negative end. As a result of the shift of the positive and negative ions, the dimensions of the material change, creating a strain within the material.

The piezoelectric coefficient is the same for both the direct and converse piezoelectric effect at the same magnitude of either electric field and induced strain or mechanical stress and induced polarization. Poisson's ratio can also play a factor into piezoelectricity; if a piezoelectric material is compressed in one direction, it may expand in the other two directions. Poisson ratio is the ratio of transverse contraction (or expansion) strain to longitudinal extension strain in the direction of stretching force. Tensile deformation is considered positive and compressive deformation is considered negative.

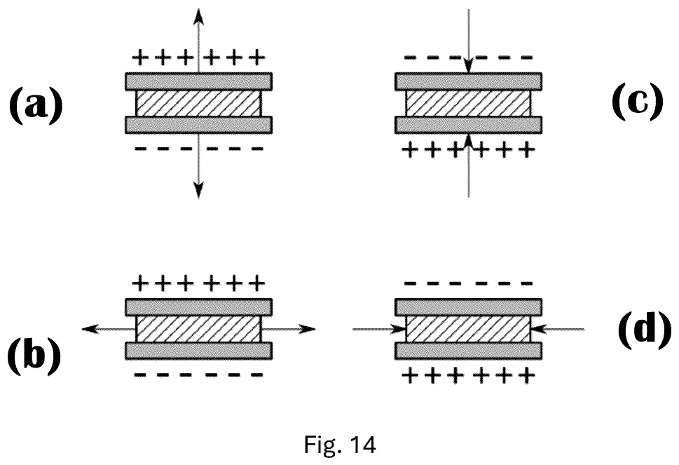

FIGS. 14a-14d illustrate the piezoelectric effect; mechanical tension or compression on a piezoelectric will induce a current or surface charge depending type of stress and direction.

Stents with Surface Enhancement Layer

Endovascular interventional procedures with implantation of stents have become one of the most frequently performed therapeutic procedures in medicine. Each year, millions of patients are treated worldwide with a variety of stent products for vascular diseases. Both drug-eluting stents and bare metal stents have been used in these interventional procedures. However, dual antiplatelet therapy (DAPT) use following intervention procedure is still needed to address the risk associated with the thrombotic risk. DAPT use associated with the bleeding risk remains as one of the major risks for patients.

The present invention provides stents that have been coated with the surface enhancement layer described hereinabove. The surface enhancement layer that contains the piezoelectric material can be deposited partially, or completely, on a stent or stent-like device. In addition, many stents have a wire-braided structure, and the following description will cover the coating or application of a surface enhancement layer on a wire-braided stent.

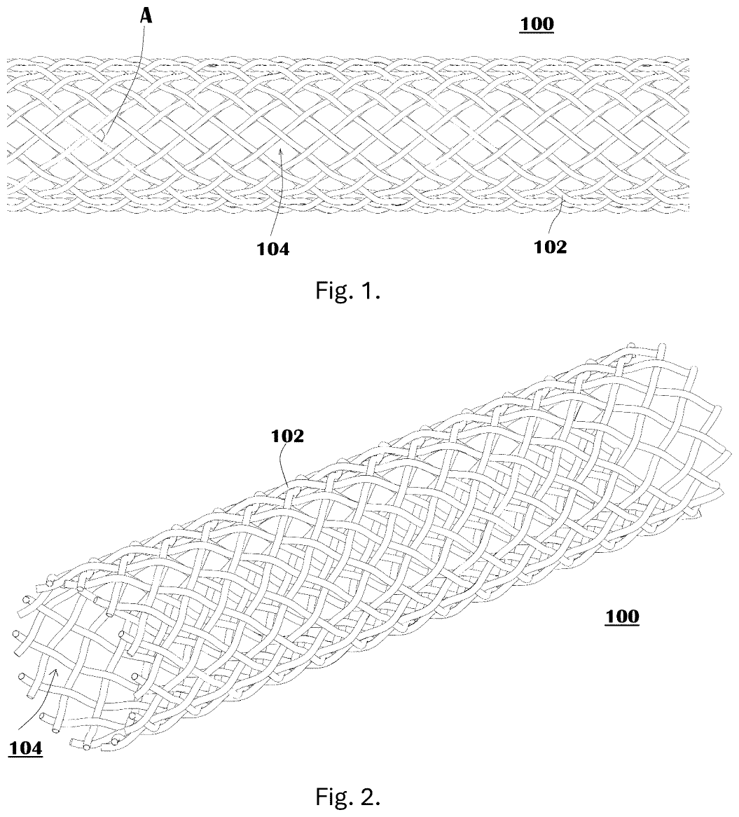

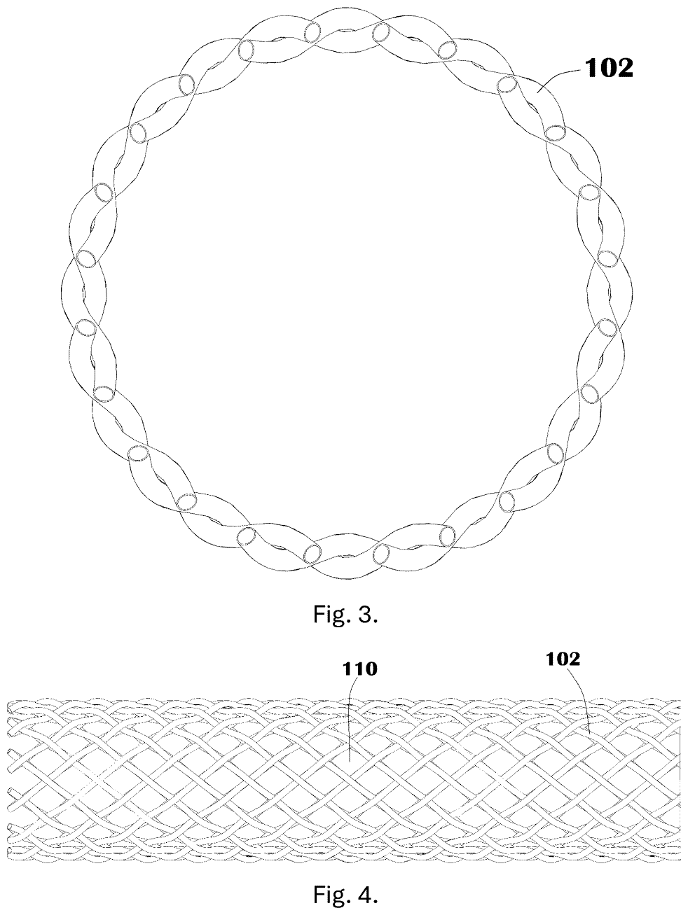

FIGS. 1-3 illustrate a wire-braided stent 100 according to one embodiment of the present invention. The stent 100 has one or more wires 102 that are braided to form a generally cylindrical structural framework that can operate as a stent. The stent 100 has generally diamond-shaped cells 104 that are formed by the braiding structure of the wires 102. Although FIGS. 1-3 illustrate a specific configuration and structure for a braided stent, this is for illustrative purposes only as the surface enhancement layer of the present invention can be applied to any cylindrical-shaped stent having any desired shape for its cells. The stent 100 shown in FIGS. 1-3 do not have the surface enhancement layer.

The surface enhancement layer can be coated or deposited in a number of different ways.

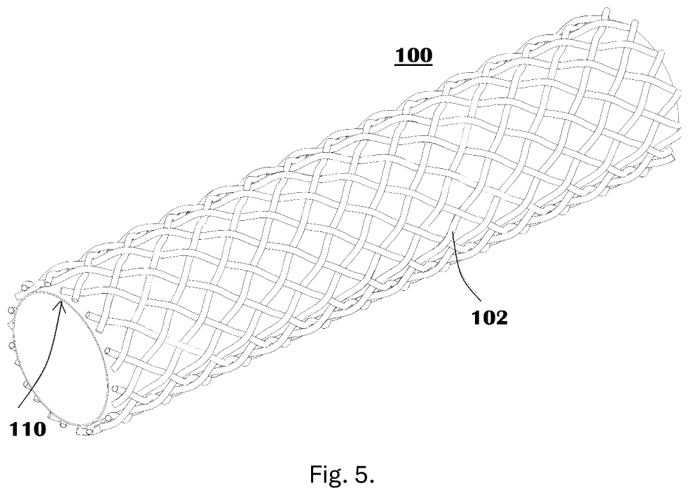

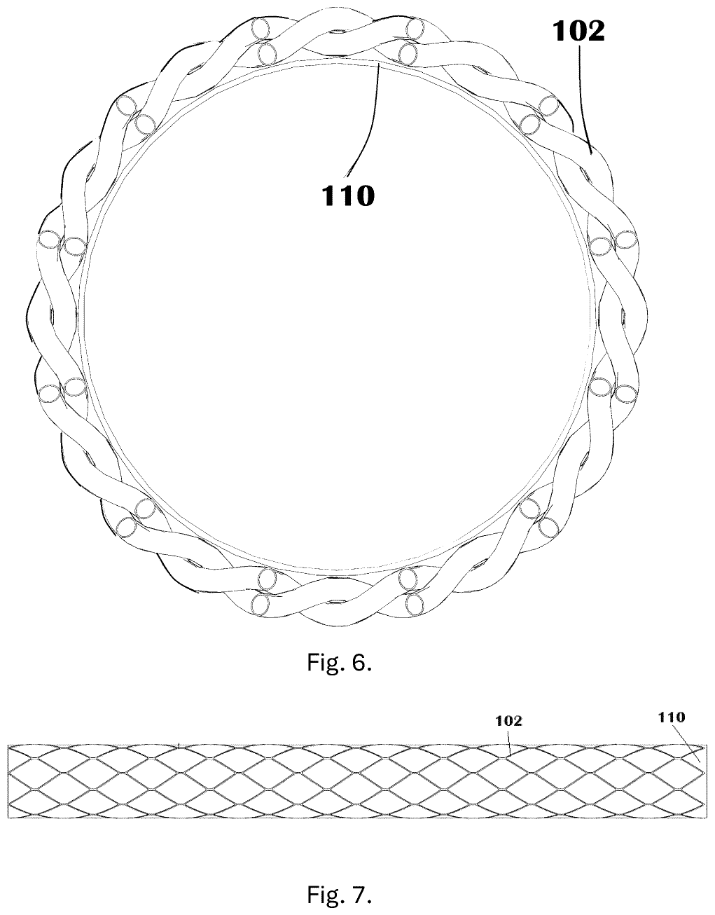

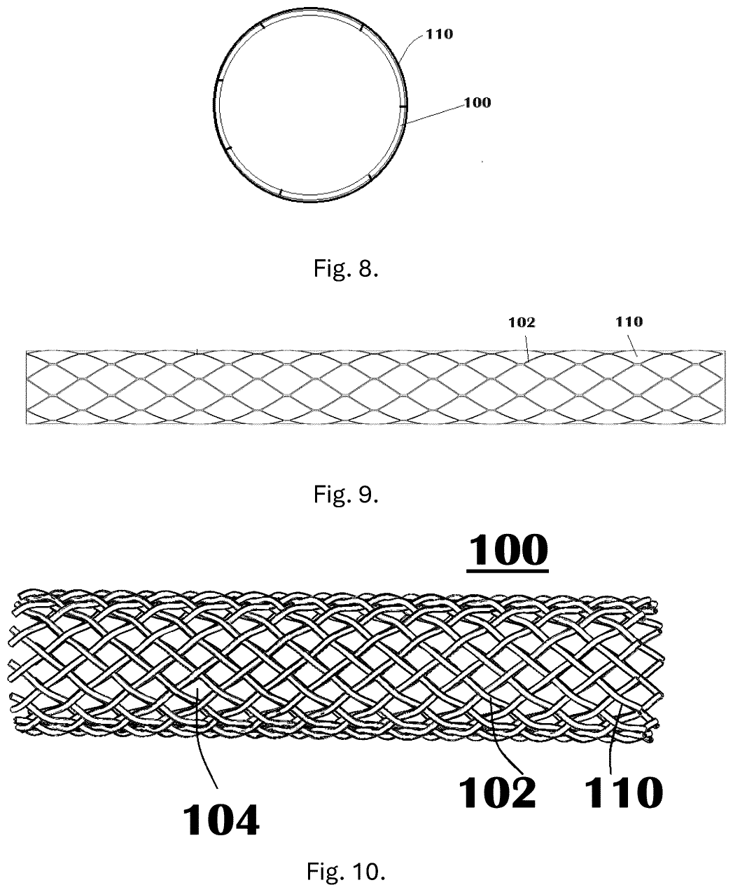

In one embodiment, as shown in FIGS. 4-6, the surface enhancement layer 110 can completely cover the inner surface of the stent 100. In another embodiment, as shown in FIGS. 7-8, the surface enhancement layer 110 can completely cover the outer surface of the stent 100. In yet another embodiment, as shown in FIG. 9, the surface enhancement layer 110 can be provided only in the cell space between the wires 102 of the stent 100. In other words, in the embodiment of FIG. 9, the surface enhancement layer 110 is not provided over the inner or outer surfaces of the stent 100. In the embodiments shown in FIGS. 4-9, there are no openings in the stent body defined by the combined surface enhancement layer 100 and stent 100 other than the openings at the opposite ends of the stent 100.

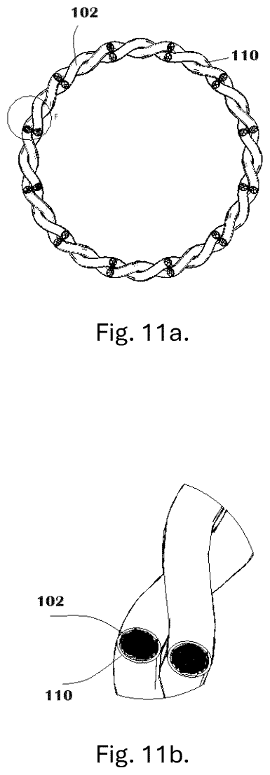

In yet another embodiment, as shown in FIGS. 10, 11A and 11B, the surface enhancement layer 110 can be applied only on the wire(s) 102, thereby leaving the entire opening in the cells 104 open. The surface enhancement layer 110 can be applied around the entire circumference of each wire 102, or along part of the circumference. For example, the surface enhancement layer 110 can be applied around the circumference of each wire 102 that defines the outer surface of the stent 100, or the inner surface of the stent 100.

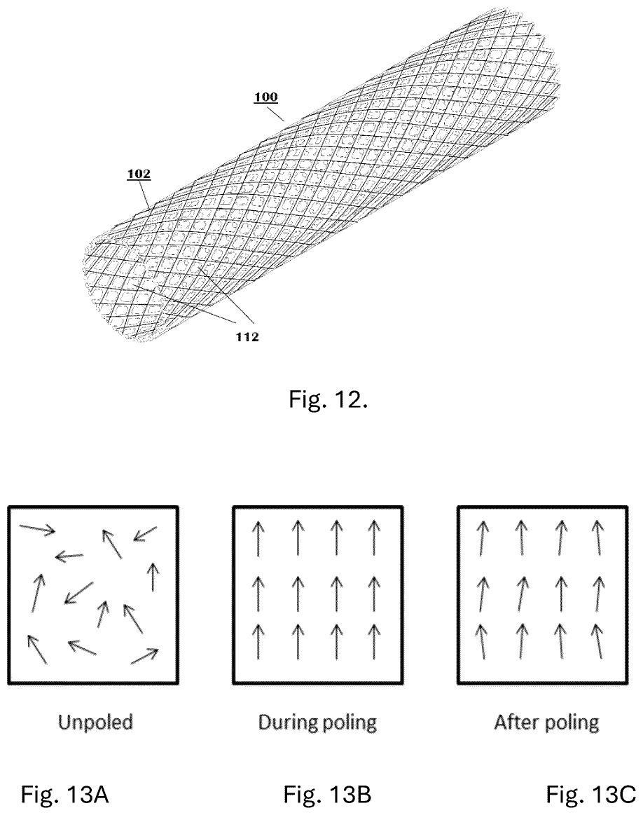

In yet another embodiment (see FIG. 12) which can be a modification of the embodiment in FIGS. 7-8, windows 112 or openings can be provided in the surface enhancement layer 110 at the locations of the cells 104. In the embodiments shown in FIGS. 10-12, there are openings in the stent body defined by the combined surface enhancement layer 110 and stent 100.

The wire-braided structure of the stent 100 can have wire counts of more than one wire, 16, 24, 32, 40, 48, 56, 64, 72, 88, 96, 144, etc. As used herein, wire count refers to the number of wires that are used to construct the entire medical device (stent 100). The wire diameter can be in the range from 0.0005″ to 0.016″. The braid angle A (see FIG. 1) can be in the range from 10 to 170 degrees. The braided stent 100 can have either opened or closed end(s), such including configurations where zero, one or both ends are closed. The materials used to construct the braid can be from a combination of Cobalt-Chromium (Co—Cr) wires plus Platinum wires, or Nitinol wires plus Platinum wires. In another embodiment, the materials to construct the braid can be a combination of Co—Cr wires plus Tantalum wires, or Nitinol wire plus Tantalum wires. In another embodiment, the materials to construct the braid can be a composite DFT (Drawn Filled Tubing) wire, such as Co—Cr wire filled with Pt or Ta, or Nitinol wire filled with Pt or Ta, etc. In yet another embodiment, the materials to construct the braid can be a combination of Co—Cr wires plus Pt—W (Platinum-Tungsten) wires, or Nitinol wires plus Pt—W wires.

In addition, the braiding angles A of the wires 102, and the porosity of the surface enhancement layer 110, can be adjusted as needed.

In one specific embodiment, the surface enhancement layer 110 can have a thickness in the range from 1 nanometer to 300 micrometers, and the pore size in the stent 100 can be in the range from 10-700 micrometers. As used herein, pore size is the size of the space or opening formed by the structure of the device, which can be the cells 104 or the windows 112. The size (surface area) of the windows 112 can be either equal or less than that of the cells 104.

In addition, different wires or sections in the braided stent 100 can have different surface enhancement layers, to control or vary the polarity or the strength of the surface charges. For example, for the braid containing Co—Cr wires plus Pt—W wires, the Co—Cr wires can be coated with a PVDF-TrFE piezoelectric material layer, while the Pt—W wires can be coated with a ZnO layer, or vice versa. Another exemplary surface layer configuration can be the combined layers from different piezoelectric material. For example, a PVDF-TrFE layer can be combined with a ZnO layer, so that one of them will be the base layer and the other one will be the top layer.

In yet another embodiment, two or more piezoelectric materials can be mixed together to be applied onto the surface of the stent 100, to optimize the thickness and bonding strength of the surface layer to the stent material, or to optimize the piezoelectric property of the surface layer. For example, the ZnO powders can be mixed in PVDF-TrFE solutions, then the surface of the stent 100 can be coated with this mixed formula in order to have the combined benefit of the materials. If P(VDF-TrFE) is used as a coating solution for dip or spray coating, the P(VDF-TrFE) needs to be dissolved in the solvents first, and then applied. If the P(VDF-TrFE) melt is used, the dilute can be used to increase the processing ability by lowing the viscosity of the P(VDF-TrFE) melt. The pore structure (windows 112) defined by the surface enhancement layer 110 can be controlled by the solvent or dilute systems during the process. For example, a stent 100 can have cells 104 that have a pore size of 200 microns, but by applying the surface enhancement layer 110 on to the struts or wires 102, the pore size will become smaller than 200 microns due to the fact that the thickness of the surface enhancement layer 110 will take up space in the cell 104, resulting in a final pore size that is smaller than the initial pore size formed by the wire or the stent struts. If the solvent makes the coating solution thicker (more viscosity), then the final pore size will be smaller; if the solvent makes the coating solution thinner (less viscosity), then the final pore size will be slightly larger.

While blended solvent or diluent systems are sometimes used in PVDF processing to achieve specific effects such as a uniform membrane pore structure, these blends are typically composed of an effective solvent and an ineffective one. In some cases, the ineffective solvent can make up as much as 95% of the blended system but nonetheless requires a small amount of effective solvent for dissolution to occur. It is worth noting that, in some cases, processing of PVDF is performed without dissolution, using non-solvents to form a slurry with a lower viscosity than that of dissolved PVDF. However, in applications such as membrane casting or gel formation, solvents or diluents play critical roles in determining the morphology and behavior of the finished product.

The solvents for P(VDF-TrFE) include:

TABLE 1
Solvents for P(VDF-TrFE) and other copolymers.
Solvent

Acetone
Acetyl triethyl citrate (ATEC)
γ-Butyrolactone (GBL)b
Cyclohexanone (CHO)
Cyclopentanone (CPO)
Dibutyl phthalate (DBP)
Dibutyl sebacate (DBS)
Diethyl carbonate (DEC)
Diethyl phthalate (DEP)
Dihydrolevoglucosenone (Cyrene)
Dimethylacetamide (DMAc)
N,N-dimethylformamide (DMF)
Dimethylsulfoxide (DMSO)
1,4-Dioxane
3-Heptanone
Hexamethyl phosphoramide (HMPA)
3-Hexanone
Methyl ethyl ketone (MEK)
N-methyl-2-pyrrolidinone (NMP)
3-Octanone
Rhodiasolv ® PolarCleana
3-Pentanone b
Propylene carbonate (PC)
Tetrahydrofuran (THF)
Tetramethylurea (TMU)
Triacetin a, b
Triethyl citrate (TEC)
Triethyl phosphate (TEP)
Trimethyl phosphate (TMP)
′tetrabutylsuccindiamide (TBSA)

The dilutes for P(VDF-TrFE):

TABLE 2
Dilutes for P(VDF-TrFE) and other copolymers.
Dilutes

Acetophenone
Acetyl tributyl citrate (ATBC)
Benzophenone
[BMIm][BF4]
ε-Caprolactam (CPL)
Cycloheptanone
Dimethyl adipate
Diethyl azelate
Diethyl glutarate a
Diethyl malonate
Diethyl oxalate
Diethyl pimelate
Diethyl succinate
Dimethyl phthalate (DMP)
Dimethyl sulfone
Diphenyl carbonate (DPC)
Ethyl benzoate (EB)
Glyceryl tributyrate (GTB)
Methyl salicylate
Sulfolane
Supercritical fluids a
Triethylene glycol diacetate (TEGDA)

The PVDF-TrFE solution for dip coating and spray coating will contain at least one of the solvents listed in the above Tables 1 and 2.

In another embodiment, primer(s) and/or adhesives can be added into the P(VDF-TrFE) in solvent solution to further improve the bonding between the medical device and the surface enhancement layer. The addition of primers and adhesives also improves the ductileness/ductility of the surface enhancement layer. One example of a primer is polyurethane or urethane-based primer such as 3M 08682 and 3M 08681. Other examples of primers include DC 1205 Primer manufactured by Dow Corning Corp., and Sofreliner PMMA-based primer manufactured by Tokuyama Corp. More examples of primers can include Silastic MDX 4-4210, Silastic Medical adhesive Type A manufactured by Dow Corning Corp., and Orto Clas manufactured by Artigos Odontologicos Classico Ltda. In other embodiments, the primers can include Lupasol P (a high-molecular weight, branched polyethyleneimine polymer) and Tyzor 217 (a water-based zirconium chelate). The percentage of the surface enhancement layer solution can contain up to 0.1 Vol % primer, 0.25% primer, 0.5% primer, 0.75% primer, 1% primer, and up to 80% primer. In another embodiment, primer(s) and/or adhesives can be applied onto the surface of the medical surface prior to the coating process in order to improve the bonding between the medical device and the surface enhancement layer.

In addition to adding the primer, biocompatible adhesive can also be added into the solvent solution or applied onto the surface of the medical device to increase the adhesion. Examples of the biocompatible adhesive include: Cyanoacrylates, Polyurethanes, and Polyesters—the resorbable aliphatic polyester poly(L-lactide) (PLLA) and Polyethylene glycol (PEG)-based adhesives, Poly-methyl Methacrylates (PMMA), and Urethane-based adhesives, etc. The percentage of the surface enhancement layer solution can contain up to 0.1 Vol % adhesive, 0.25% adhesive, 0.5% adhesive, 0.75% adhesive, 1% adhesive, and up to 80% adhesive.

In addition, a top coat may be applied over the surface enhancement layer. The top coat usually is a thin dielectric layer, and is not conductive. The top coat layer is used to prevent the release of the surface charge. Otherwise, without a top coat, the surface charge can induce current in the conductive blood environment. With the top coat, which acts like a capacitor, the surface charge generated by the surface layer can stay and form the electric field on the surface of the medical device to control the blood-device interaction.

The thickness of the top coat can be in the range from 1-200 nanometers. The materials for the top coat can either be dielectric biocompatible polymers such as parylene, PTFE, PTFE-like coating, hydrophobic coating, hydrophilic coating, etc. The top coat can also be metallic oxides, such as TaO, TiO, ZnO, ZrO, etc., in order to maintain the surface electric charges from the surface enhancement layer. The coating processes include plasma coating, 3D printing, 3D printed coating, chemical vapor deposition (CVD), plasma enhanced CVD (PE-CVD), dip coating, spray coating, ultrasound spray coating, ion beam deposition, sputtering coating, electric spinning, vacuum deposition, physical vapor deposition (PVD), hot dip galvanized zinc (HDG) coating, coating by electroplating, atomic layer deposition (ALD), etc.

As described above, in one embodiment, a ZnO coating can be applied on the medical device to act as the piezoelectric surface enhancement layer. ZnO is a piezoelectric material, and can generate surface electric charge under stress/deformation. The ZnO layer can be formed by immersing the stent 100 in a bath of molten Zinc at a temperature of around in the range between 200-450° C. When exposed to the atmosphere, the pure zinc (Zn) on the surface of the device reacts with oxygen (O2) to form zinc oxide (ZnO). The typical hot dipping process to form the ZnO layer on device surface includes:
A. Clean and activate the device surface using a caustic solution.
B. Rinse off the caustic cleaning solution.
C. A flux, often zinc ammonium chloride, is applied to the device surface to inhibit oxidation of the cleaned surface upon exposure to air. The flux is allowed to dry on the device and aids in the process of the liquid zinc wetting and adhering to the device. This process step is optional.
D. The device is dipped into the molten zinc bath and held there until the temperature of the device equilibrates with that of the bath.
E. The device is cooled in a quench tank to reduce its temperature and inhibit undesirable reactions of the newly formed coating with the atmosphere.

In another embodiment, ZnO can be mixed with a polymer or a piezoelectric polymer to be coated on the surface of the medical device. The percentage of the ZnO in the polymer or piezoelectric material solution can be in the range from 0.1% to 36% by weight. ZnO can be in fine powder or liquid form.

Other processes that can be used to apply the ZnO surface layer include:
A. Chemical Vapor Deposition (CVD): This method involves depositing gaseous reactants onto a substrate, where they react or decompose to form the desired ZnO layer.
B. Physical vapor deposition.
C. Sputtering: In this technique, a target material (in this case, zinc) is bombarded with high-energy particles, causing atoms to be ejected and deposited as a thin film on a substrate.
D. Sol-Gel Process: This involves the transition of a solution system from a liquid “sol” (mostly colloidal) into a solid “gel” phase. In the case of ZnO, zinc-containing solutions are used to form a gel that is then dried and heat-treated to form a ZnO layer.
E. Thermal Oxidation: Zinc metal or zinc films are oxidized at high temperatures in the presence of oxygen to form ZnO.
F. Aqueous Chemical Growth: This process involves the growth of ZnO nanostructures from aqueous solutions. It typically starts with a seed layer of ZnO nanoparticles on a substrate, which facilitates the nucleation and growth of ZnO nanostructures upon heating.
G. Electrochemical Deposition: This method uses an electric current to reduce zinc ions in a solution to zinc metal, which then reacts with oxygen to form ZnO.
H. Atomic layer deposition (ALD)
I. Wet Oxidation

Once the surface enhancement layer 110 is applied to the surface of the medical device, the polarity of the device can be set or controlled using a poling process. During the poling process, the coated and cleaned device is placed in a strong electric field for a certain amount of time. After poling, the surface enhancement layer acquires the desired piezoelectric properties. The polarity of the device, which is the polarity of the coated surface (either inner or outer surface or both) under mechanical deformation, can be controlled by the poling process. For example, the device can be poled to have a positive surface charge on the outer surface of the device, while the inner surface of the device can have negative surface charge; and vice versa. For implants in the main parent vessel, the blood contact surface needs to generate a negative surface charge under mechanical deformation. For implants used to occlude the vessel or an aneurysm, the blood contact surface needs to generate a positive surface charge under mechanical deformation.

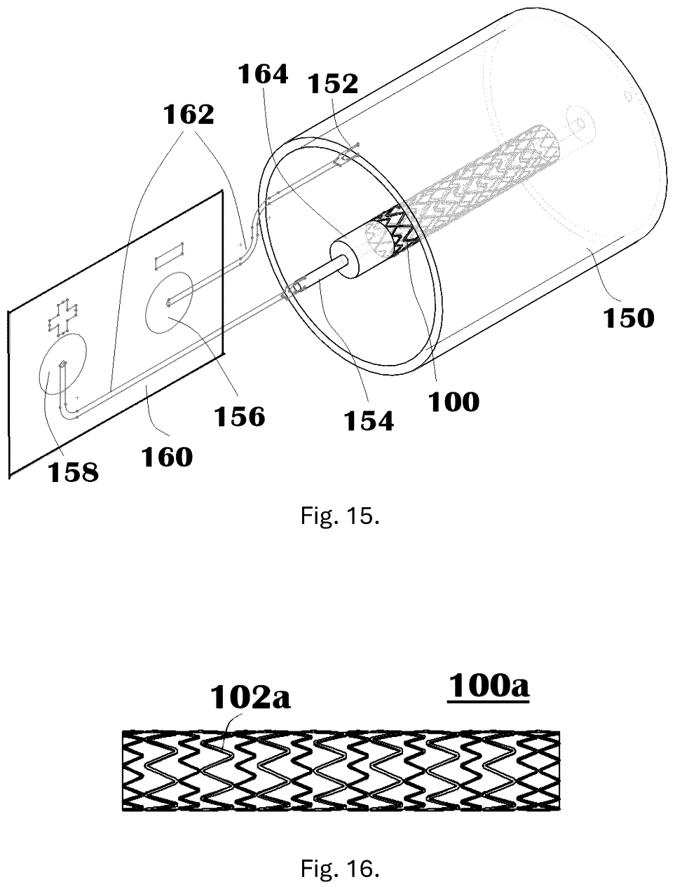

FIG. 15 illustrates the poling apparatus for poling the coated stent 100 of FIGS. 4-12. It can be used to pole laser-cut stents, braided stents, braided stent-like devices, and any other devices with a cylindrical shape having a lumen or similar space inside the device.

A generally cylindrical housing 150 is provided to retain the stent 100 (i.e., the device to be poled) inside during the application of the electric field. A first electrode 152 and a second electrode 154 are provided. The first electrode 152 is electrically coupled via electric cable 162 to a negative output port 156 of a power supply 160, and has a connection that is coupled to the wall of the housing 150. The second electrode 154 has one end that is electrically coupled via electric cable 162 to a positive output port 158 of the power supply 160, and has an opposite end that is received inside a spacer 164.

More specifically, the first electrode 152 has a circular-shaped structure made from conductive metal material. The diameter can be between 0.5 mm to 60 mm; the thickness can be from 0.005 mm to 20 mm; and the length can be from 0.5 mm to 500 mm. The first electrode 152 can either be the positive electrode or negative electrode.

The second electrode 154 is a circular-shaped rod or tubing made from conductive metal materials. Its diameter can be between 0.05 mm to 55 mm. If it is in a tubing configuration, the wall thickness can be between 0.005 mm to 2 mm. The length can be from 0.5 mm to 500 mm. The second electrode 154 can either be the positive electrode or negative electrode.

The spacer 164 is made from non-conductive material, such as polymers. The length of the spacer 164 can be between 0.5 mm to 500 mm. The thickness of the spacer 164 can be between 0 mm (i.e., no spacer) to 50 mm.

The method for poling includes the following steps:

Step 1: Load the stent 100 onto the second electrode 154, so that the two ends of the stent 100 are supported by the spacer 164, and the entire length of the stent 100 does not touch the second electrode 154. The spacer 164 can either cover the entire length of the second electrode 154, or only cover a portion of the second electrode 154. For example, the spacer 164 can have a continuous configuration, to cover the full length or a portion of the length of the second electrode 154; or the spacer 164 can have discontinued configuration, being comprised of more than one piece. For example, one short spacer piece can be provided at each end of the stent 100 to support the stent 100 only from the two ends of the stent to avoid direct contact with the second electrode 154. Alternatively, one or more pieces of spacer 164 can be added on to the second electrode 154 near the middle portion of the stent 100, to provide extra support to the stent 100 so that stent 100 will not contact with the second electrode 154. If the stent 100 is longer in length, two or more pieces of spacer 164 may be needed. When the thickness of the spacer is zero, then the stent 100 will be in direct contact with the second electrode 154.

Step 2: Connect both first electrode 152 and second electrode 154 to the power supply 160.

Step 3: Turn on the power supply 160 and hold the voltage (which can be in the range from 2,000 volts to 60,000 volts) for a certain duration (e.g., from 1 to 240 minutes).

Step 4: Once the desired time is reached, turn off the power supply 160, and remove the stent 100 from the second electrode 154.

The polarity on the stent 100 and the type of charge (+ or −) on the surface of the stent 100 (when it is deformed) can be controlled by the poling process. For example, if it is desired to have a positive charge on the outer surface of the stent 100, then the first electrode 152 can be connected with the negative electrode on the power supply 160, while the second electrode 154 can be connected with the positive electrode on the power supply 160. With these connections, the stent polarity will be as follows: the outer surface will have a positive charge, and the inner surface will have a negative charge when the stent 100 is deformed. Conversely, if it is desired to have a negative charge on the outer surface, then the first electrode 152 can be connected with the positive electrode on the power supply 160, while the second electrode 154 can be connected with the negative electrode on the power supply 160. With these connections, the stent polarity will be as follows: the outer surface will have a negative charge, and the inner surface will have a positive charge when the stent 100 is deformed.

The principles of the present invention are described hereinabove in connection with a laser cut stent 100, but the same principles can be applied to any stent, including braided stents made from wires, as shown in FIGS. 1-12.

Embolic Coils with Surface Enhancement Layer

Embolic Coils are used to occlude the body lumen and vessel abnormalities, such as aneurysms, and leakages in the vessel or other body lumens. There are many embolic coil products are on the market, but the unmet clinical needs still exist, which mainly include incomplete occlusion, delayed full occlusion, and poor healing effect (e.g., luminal interface are not covered by endothelium).

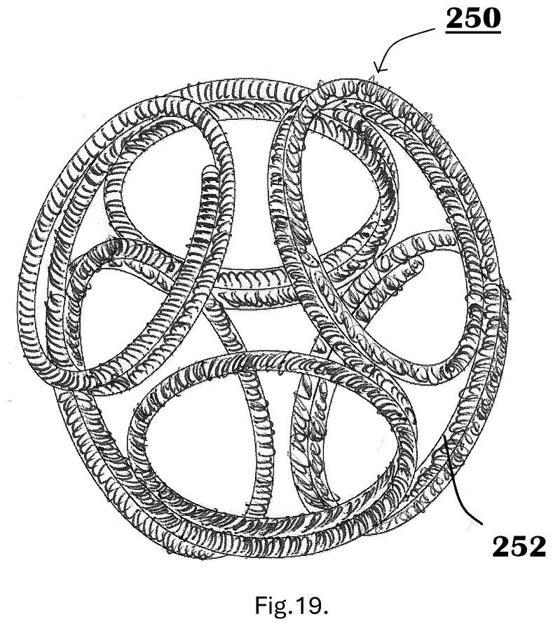

FIGS. 18 and 19 illustrate the underlying structural framework for two conventional types of embolic coils.

Integrating the surface enhancement layer of the present invention on the structural framework of the embolic coils can alter their interaction with blood. For example, with the coil's outer surface (blood contact surface) coated with a piezoelectric coating, positive surface charges can be generated on the coil surface during movement of the coil. Since most of the blood components carry negative surface charge, when contact is made with the positively-charged coil surface, the blood components can be attracted and adhere to the coil surface, thereby promoting thrombosis formation, and leading to speedy occlusion and endothelium growth at the interface.

The principles and methods described hereinabove for the stents 100 can also be applied to embolic coils. The materials used for the embolic coils include Pt, Pt—W, Pt—Ir, and similar materials. The surface enhancement layer can be fully or partially coated on the surface of the embolic coils. The top coat made from dielectric materials can also be coated on top of the surface enhancement layer if preferred.

Different sections on the embolic coil can have different surface enhancement layers. For example, one section of the embolic coil can have a P(VDF-TrFE) piezoelectric coating, and another section can have a ZnO piezoelectric coating. The different coating sections can vary along the length or circumference of the embolic coil.

After the poling process, when the embolic coil is deformed, its outer surface can have a positive charge which promotes the formation of thrombosis over the embolic coil.

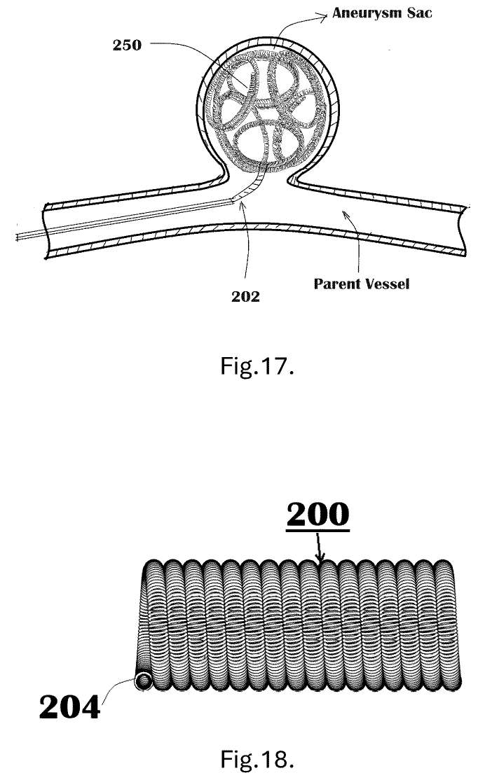

FIG. 17 illustrates a three-dimensional (3D) embolic coil 250 that has been delivered to an aneurysm sac by a delivery device 202 (e.g., a microcatheter). The 3D embolic coil 250 is shown in FIG. 19. The 3D embolic coil 250 includes the surface enhancement layer 252. The 3D embolic coil 250 has a somewhat spherical structure when it is released from the delivery catheter.

FIG. 18 illustrates a 2D embolic coil 200 that includes the surface enhancement layer 204. The 2D embolic coil 200 has a helix or spring-like structure when it is released from delivery catheter.

The embolic coils 200 and 250 that are shown in FIGS. 18 and 19 are illustrative only, as the principles of the present invention can be applied to embolic coils having any desired configuration or structure.

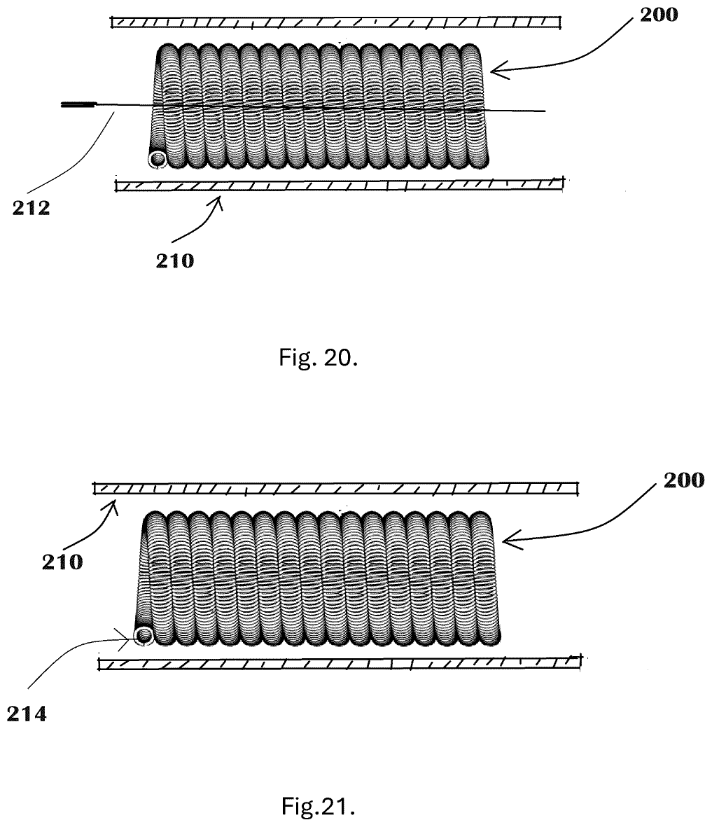

FIG. 20 illustrates an apparatus for poling the 2D embolic coil 200 in FIG. 18. The apparatus includes a first electrode 210 that is shaped as a cylinder and is positioned outside the embolic coil 200. A second electrode 212 is configured as a single wire and extends through the internal lumen formed by the geometric coiling (helix) of the embolic coil 200. Each of the first electrode 210 and the second electrode 212 can be positive (+) or negative (−). The poling process is similar to the process described hereinabove for the stents, where high voltage can be applied through the electrodes 210 and 212 to polarize the surfaces (outer and inner) of the embolic coil 200 that have been coated with the surface enhancement layer.

FIG. 21 illustrates another apparatus for poling the 2D embolic coil 200 in FIG. 18. The first electrode 210 can be the same as in the apparatus in FIG. 20, and is shaped as a cylinder and is positioned outside the embolic coil 200. The positioning of the second electrode 214 is different from that in FIG. 20, as this second electrode 214 is the coil wire (which is made of metallic material) itself that is used for the embolic coil 200. Thus, the coil wire for the embolic wire 200 will be used as another electrode to polarize the surface enhancement layer 204 on embolic coil 200 itself.

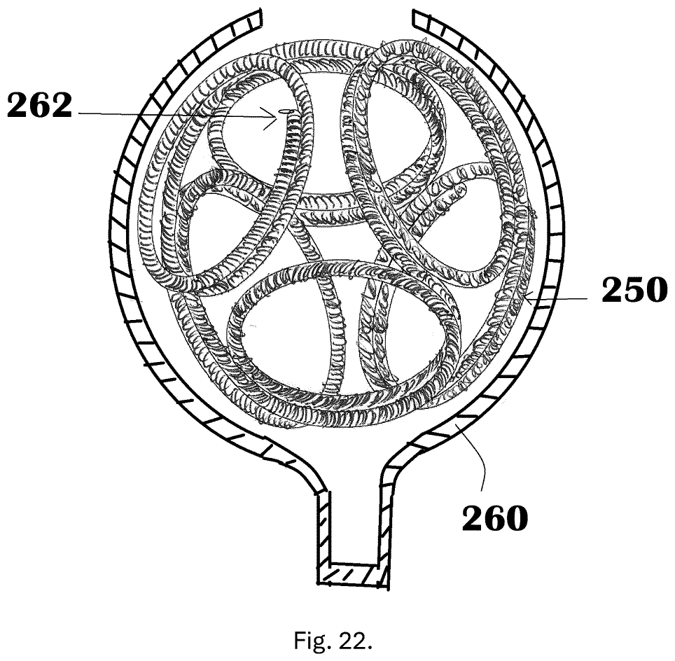

FIG. 22 illustrates an apparatus for poling the 3D embolic coil 250 in FIG. 19. The apparatus includes a first electrode 260 that is cylindrical or spherical in shape to be positioned outside of (i.e., surrounds) the embolic coil 250. The coil wire itself will act as the second electrode 262.

For the apparatus in FIGS. 20-22, the voltage range for poling can be from 2,000V to 60,000V. In addition, the distance between the two electrodes can be in the range from 0.1 mm to 50 mm.

### Example 1

A braided vascular device (such as the stent 100 shown in FIG. 10) was fabricated as follows. The stent 100 contains 64 metal wires with a diameter of approximately 0.0015″ (Base Materials), of which 16 were Pt (90%) alloyed with W (10%) wires, 16 were L605 Co—Cr alloy wires, and 32 were Nitinol superelastic wires. The stent 100 has an outside diameter of 4 mm and the PPI along the longitudinal direction of 260, with a braid angle A of approximately 70 degrees. PPI means “Picks Per Inch” in braiding and refers to the number of times a wire crosses over one inch.

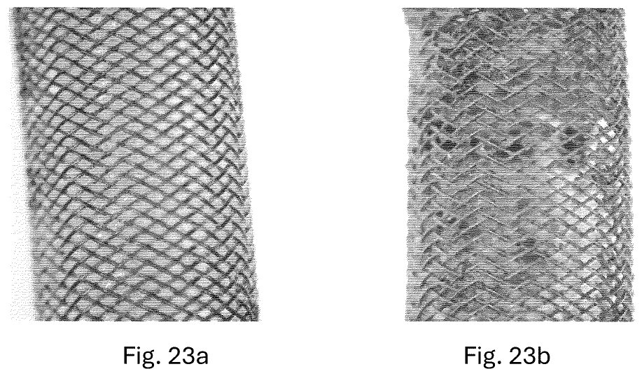

Once braided, the stent 100 is cut to a length of 20 mm, and cleaned ultrasonically in ethanol solution bath. The cleaned “bare” braided device then is prepared as follows:
1. Surface activation: The surface of each stent 100 is activated in an acid solution with a composition as HF:HNO3:H2O ratio (volume %) 1:5:20 for 1 minute with ultrasonic agitation. Then the stent 100 is rinsed in ethanol, followed by a rinse in deionized water (DI) water.
2. Surface enhancement layer solution preparation: Mix P(VDF-TRFE) with Methyl ethyl ketone (MEK) (Solvent) so that the concentration of the solution is approximately 2% P(VDF-TrFE). Stir the solution in mixer at 2000 rpm for 60 minutes.
3. Coating and annealing processes: The stent 100 is dipped into the surface enhancement layer solution for 2 minutes and annealed at 138 degrees Celcius for 45 minutes to enhance the crystallinity of the P9VDF-TrFE) crystal structure for improved piezoelectric property, and increased beta phase in the crystal structure of the P(VDF-TrFE) material. Beta phase in the crystal structure of the P(VDF-TrFE) material is responsible for the piezoelectric property, so an increased beta phase structure means a stronger piezoelectric property. The annealing temperature (Ta) range can be between 80 to 165 degrees Celcius, a temperature between the Glass transition temperature (Tg) and the melting temperature (Tm) of the P(VDF-TrFE) polymer, that is, Tg<Ta<Tm. The annealing duration can be in the range from 5 minutes to 24 hours. The surface enhancement layer has gained some initial piezoelectric property after the annealing process.
4. Poling Process: After the surface enhancement layer coating and annealing processes, the stent 100 is then polarized through the poling process to define the polarity of the device. As shown in FIG. 15, the stent 100 is loaded onto the electrode 154; and the cylindrical shaped electrode 150 is positioned over the stent 100. The inner diameter of the electrode 150 is 4.5 mm. The inner diameter of the electrode 150 can be in the range between 2 mm to 20 mm depending on the diameter of the stent 100 to be poled. The electrodes 154 and 150 are connected to the power supply through the cables, and the power supply is turned on. The output voltage is set to 3000 volts and power kept on for 30 minutes. The 3000 volts is used for this specific embodiment, but the voltage range for poling process can be a voltage in the range from 2000 volts to 60 Kv. The duration used in this specific embodiment is 30 minutes, but the duration can be vary in the range between 2 minutes to 3 hours. For the finished braided vascular device, polarization needs to be done in a way that, under the deformation, the inner surface generates the negative charge, while in outer surface generates the positive charge. Therefore, during the poling process, the electrode 154 needs to be connected to the “+” output port, while the electrode 150 need to be connected to the “−” output port of the power supply. Vice versa, if the electrode 154 is connected to the “−” output port of the power supply, electrode 150 is connected with the “+” output port of the power supply, such that the resulting device will have the opposite polarity, which is, under deformation, the inner surface having a positive charge and the outer surface having a negative charge.
5. The stent 100 with surface enhancement layer having different polarization is then tested for the controlled thrombogenicity through a bench “Chandler Blood Loop Testing”. A Chandler loop system enables simulation of extracorporal blood circulation (ECC). Polymer tubes, partly filled with blood, are formed into re-closable loops and are rotated with a rotation speed between 10-100 RPM in a temperature-controlled environment, to simulate arterial flow conditions. This device offers an experimental platform for testing interactions of blood with artificial materials and surfaces, their biocompatibility or, to be precise, hemocompatibility and hemorheological effects.
6. The stent 100 with different polarity is placed in a 4 mm plastic tubing. The tubing is filled with fresh animal blood. The plastic tubing with the stent 100 are then loaded onto the Chandler Loop Tester. The Chandler Loop test was run at body temperature for 120 minutes with 100 rpm speed. FIGS. 23A and 23B show the test results, which came out as expected. Specifically, for the device shown in FIG. 23A, under the deformation or the shear force from the blood flow, the inner surface had negative surface charge, while the outer surface had positive surface charge. As a result of these controlled polarities, the inner surface of this device is anti-thrombosis. For the device shown in FIG. 23B, under the deformation or the shear force from the blood flow, the inner surface has a positive surface charge, while the outer surface has a negative surface charge. As a result of these controlled polarity, the inner surface of this device promotes the formation of the thrombosis.

### Example 2

A braided vascular device (such as the stent 100 shown in FIG. 10) was fabricated as follows. The stent 100 contains 64 metal wires, each with a diameter of approximately 0.0015″ (Base Materials), of which 16 were Pt (90%) alloyed with W (10%) wires, 16 were L605 Co—Cr alloy wires, 32 were Nitinol super elastic wires. The stent 100 has an outside diameter of 4 mm and a PPI along the longitudinal direction of 26, with a braid angle A of approximately 70 degrees.

Once braided, the braided stent 100 is cut to a length of 20 mm, and cleaned ultrasonically in ethanol solution bath. The cleaned “bare” braided device then is prepared as follows:
1. Surface activation: The surface of each stent 100 is activated in an acid solution with a composition as HF:HNO3:H2O ratio (volume %) 1:5:20 for 1 minute with ultrasonic agitation. Then the stent 100 is rinsed in ethanol, followed by rinsing in DI water.
2. Surface enhancement layer solution preparation: Mix P(VDF-TRFE) with Methyl ethyl ketone (MEK) (solvent) so that the concentration of the solution is approximately 2% P(VDF-TrFE). In order to enhance the crystallinity of the P(VDF-TrFE) polymer, 1% of ZnO nanoparticle is added into the solution as nucleating agents to generate the required nucleation sites, and also to significantly accelerate the crystallization rate of the polymer. In addition, ZnO itself is a material that exhibits piezoelectric properties under the deformation. The solution is stirred in mixer at 2000 rpm for 60 minutes. In addition to ZnO nanoparticles, BaTiO3, TiO2, carbon black (CB), single-walled carbon nanotubes (SWCNT), and graphene oxide (GO) can be added into the solution for a piezoelectric properties.
3. Coating and annealing processes: The stent 100 is then dipped into a surface enhancement layer solution for 2 minutes and annealed at 138 degrees Celcius for 45 minutes to enhance the crystallinity of the P9VDF-TrFE) crystal structure for improved piezoelectric property, and increased beta phase in the crystal structure of the P(VDF-TrFE) material. The annealing temperature (Ta) range can be between 80 to 165 degrees Celsius, a temperature between the glass transition temperature (Tg) and the melting temperature (Tm) of the P(VDF-TrFE polymer, that is, Tg<Ta<Tm. The annealing duration can be in the range from 5 minutes to 24 hours. The surface enhancement layer has now gained some piezoelectric property after the annealing process.
4. Poling Process: After the surface enhancement layer coating and annealing processes, the stent 100 is then polarized through the poling process to define the polarity of the device. As shown in FIG. 15, the stent 100 is loaded onto the electrode 154, and cylindrical shaped electrode 150 is placed over the stent 100. The inner diameter of the electrode 150 is 4.5 mm. The inner diameter of the electrode 150 can be in the range between 2 mm to 20 mm depending on the diameter of the device to be poled. The electrodes 154 and 150 are connected to the power supply through the cables. The power supply is turned on, and the output voltage is set to 3000 volts and kept for 30 minutes, although the ranges noted in Example 1 can also apply. For the finished stent 100, polarization needs to be done in a way that, under the deformation, the inner surface generates a negative surface charge, while the outer surface generates a positive charge. Therefore, during the poling process, the electrode 154 needs to be connected to the “+” output port, while the electrode 150 needs to be connect to the “−” output port of the power supply. Vice versa, if the electrode 154 is connected to the “−” output port of the power supply, the electrode 150 is connected with the “+” output port of the power supply, so that the resulting device will have opposite polarity, which is, under deformation, the inner surface has positive charge and the outer surface has negative charge.

### Example 3

A braided vascular device (such as the stent 100 shown in FIG. 10) was fabricated as follows. The stent 100 contains 64 metal wires, each with a diameter of approximately 0.0015″ (Base Materials), of which 16 were Pt (90%) alloyed with W (10%) wires, 16 were L605 Co—Cr alloy wires, and 32 were Nitinol superelastic wires. The stent 100 has an outside diameter of 4 mm and a PPI along the longitudinal direction of 260, and with a braid angle A of approximately 70 degrees.

Once braided, the braided stent 100 is cut to a length of 20 mm, and cleaned ultrasonically in ethanol solution bath. The cleaned “bare” braided device then is prepared as follows:
1. Surface activation: The surface of each stent 100 is activated in an acid solution with a composition as HF:HNO3:H2O ratio (volume %) 1:5:20 for 1 minute with ultrasonic agitation. Then the braided device is rinsed in ethanol, followed by rinsing in DI water.
2. The clean stent 100 is then dipped into a 3M 08682 Primer for 45 seconds, pulled out, and cured at 80 degrees Celcius for 5 minutes.
3. Surface enhancement layer solution preparation: Mix P(VDF-TRFE) with Methyl ethyl ketone (MEK) (solvent) so that the concentration of the solution is approximately 2% P(VDF-TrFE). In order to enhance the crystallinity of the P(VDF-TrFE) polymer, 1% of ZnO nanoparticle is added into the solution as nucleating agents to generate the required nucleation sites, and also to significantly accelerate the crystallization rate of the polymer. In addition, ZnO itself is a material that exhibits piezoelectric properties under deformation. The solution is stirred in mixer at 2000 rpm for 60 minutes. In addition to ZnO nanoparticles, BaTiO3, TiO2, carbon black (CB), single-walled carbon nanotubes (SWCNT), and graphene oxide (GO) can also be added into the solution for piezoelectric purposes.
4. Coating and annealing processes: The stent 100 is dipped into the surface enhancement layer solution for 2 minutes and annealed at 138 degrees Celcius for 45 minutes to enhance the crystallinity of the P9VDF-TrFE) crystal structure for improved piezoelectric property, and increased beta phase in the crystal structure of the P(VDF-TrFE) material. The annealing temperature (Ta) range can be between 80 to 165 degrees Celcius, a temperature between the glass transition temperature (Tg) and the melting temperature (Tm) of the P(VDF-TrFE polymer, that is, Tg<Ta<Tm. The annealing duration can be in the range from 5 minutes to 24 hours. The surface enhancement layer has now gained some piezoelectric properties after the annealing process.
5. Poling Process: After the surface enhancement layer coating and annealing processes, the stent 100 is then polarized through the poling process to define the polarity of the device. As shown in FIG. 15, the stent 100 is loaded onto the electrode 154, and the cylindrical shaped electrode 150 is positioned over the stent 100. The inner diameter of the electrode 150 is 4.5 mm. The inner diameter of the electrode 150 can be in the range between 2 mm to 20 mm depending on the diameter of the device to be poled. The electrodes 154 and 150 are connected to the power supply through the cables/The power supply is turned on and the output voltage is set to 3000 volts and kept for 30 minutes. For the finished stent 100, polarization needs to be done in a way that, under deformation, the inner surface generates a negative surface charge, while the outer surface generates a positive charge. Therefore, during the poling process, the electrode 154 needs to be connected to the “+” output port, while the electrode 150 needs to be connect to the “−” output port of the power supply. Vice versa, if the electrode 154 is connected to the “−” output port of the power supply, the electrode 150 is connected with the “+” output port of the power supply, so that the resulting device will have opposite polarity, such that, under deformation, the inner surface has positive charge and outer surface has negative charge.

### Example 4

A braided vascular device (such as the stent 100 shown in FIG. 10) was fabricated as follows. The stent 100 contains 64 metal wires, each with a diameter of approximately 0.0015″ (Base Materials), of which 16 were Pt (90%) alloyed with W (10%) wires, 16 were L605 Co—Cr alloy wires, 32 were Nitinol superelastic wires. The stent 100 has an outside diameter of 4 mm and a PPI along the longitudinal direction of 260, with a braid angle A of approximately 70 degrees.

Once braided, the stent 100 is cut to length of 20 mm, and cleaned ultrasonically in ethanol solution bath. The cleaned “bare” braided device then is prepared as follows:
1. Surface activation: The surface of each stent 100 is activated in an acid solution with a composition as HF:HNO3:H2O ratio (volume %) 1:5:20 for 1 minute with ultrasonic agitation. Then the braided device is rinsed in ethanol, followed by rinsing in DI water.
2. Surface enhancement layer solution preparation: Mix P(VDF-TRFE) with Methyl ethyl ketone (MEK) (solvent) so that the concentration of the solution is approximately 2% P(VDF-TrFE). In order to enhance the crystallinity of the P(VDF-TrFE) polymer, 1% of ZnO nanoparticle is added into the solution as nucleating agents to generate the required nucleation sites, and to also significantly accelerate the crystallization rate of the polymer. Also, ZnO itself is a material that exhibits piezoelectric properties under deformation. 0.5% 3M 08682 Primer is added into the solution. The solution is stirred in a mixer at 2000 rpm for 60 minutes. In addition to ZnO nanoparticles, BaTiO3, TiO2, carbon black (CB), single-walled carbon nanotubes (SWCNT), and graphene oxide (GO) can also be added into the solution for piezoelectric purposes.
3. Coating and annealing processes: The stent 100 is dipped into surface enhancement layer solution for 2 minutes and annealed at 138 degrees Celsius for 45 minutes to enhance the crystallinity of the P9VDF-TrFE) crystal structure for improved piezoelectric property, and increased beta phase in the crystal structure of the P(VDF-TrFE) material. The annealing temperature (Ta) range can be between 80 to 165 degrees Celsius, a temperature between the Glass transition temperature (Tg) and the melting temperature (Tm) of the P(VDF-TrFE polymer, that is, Tg<Ta<Tm. The annealing duration can be in the range from 5 minutes to 24 hours. The surface enhancement layer has gained some piezoelectric properties after the annealing process.
4. Poling Process: After the surface enhancement layer coating and annealing processes, stent 100 is then polarized through the poling process to define the polarity of the device. As shown in FIF. 15, the stent 100 is loaded onto the electrode 154, and the cylindrical shaped electrode 150 is positioned over the stent 100. The inner diameter of the electrode 150 is 4.5 mm. The inner diameter of the electrode 150 can be in the range between 2 mm to 20 mm depending on the diameter of the stent to be poled. The electrodes 154 and 150 are connected to the power supply through the cables. The power supply is turned on, and the output voltage to set to 3000 volts and kept for 30 minutes. The voltage can be another value in the range from 2000 volts to 50 Kv as well, and the duration can be another value in the range between 2 minutes to 3 hours as well. For the finished stent 100, polarization needs to be done in a way that, under the deformation, the inner surface generates a negative surface charge, while the outer surface generates a positive charge. Therefore, during the poling process, the electrode 154 needs to be connected to the “+” output port, while the electrode 150 needs to be connect to the “−” output port of the power supply. Vice versa, if the electrode 154 is connected to the “−” output port of the power supply, the electrode 150 is connected with the “+” output port of the power supply, so that the resulting device will have opposite polarity such that, under deformation, the inner surface has positive charge and outer surface has negative charge.

### Example 5

A braided vascular device (such as the stent 100 shown in FIG. 10) was fabricated as follows. The stent 100 contains 64 metal wires, each with a diameter of approximately 0.0015″ (Base Materials), of which 16 were Pt (90%) alloyed with W (10%) wires, 16 were L605 Co—Cr alloy wires, 32 were Nitinol superelastic wires. The stent 100 has an outside diameter of 4 mm and the PPI along the longitudinal direction of 260, with a braid angle A of approximately 70 degrees.

Once braided, the stent 100 is cut to a length of 20 mm, and cleaned ultrasonically in ethanol solution bath. The cleaned “bare” braided device then is prepared as follows:
1. Surface activation: The surface of each stent 100 is activated in an acid solution with a composition as HF:HNO3:H2O ratio (volume %) 1:5:20 for 1 minute with ultrasonic agitation. Then the braided device is rinsed in ethanol, followed by rinsing in DI water.
2. Surface enhancement layer solution preparation: Mix P(VDF-TRFE) with Methyl ethyl ketone (MEK) (Solvent) so that the concentration of the solution is approximately 2% P(VDF-TrFE). In order to enhance the crystallinity of the P(VDF-TrFE) polymer, 1% of ZnO nanoparticle is added into the solution as nucleating agents to generate the required nucleation sites, which also significantly accelerates the crystallization rate of the polymer. In addition, ZnO itself is a material that exhibits piezoelectric properties under deformation. The solution is stirred in a mixer at 2000 rpm for 60 minutes. In addition to ZnO nanoparticles, BaTiO3, TiO2, carbon black (CB), single-walled carbon nanotubes (SWCNT), and graphene oxide (GO) can be added into the solution for piezoelectric purposes.
3. Coating and annealing processes: the stent 100 is dipped into surface enhancement layer solution for 2 minutes and annealed at 138 degrees Celcius for 45 minutes to enhance the crystallinity of the P9VDF-TrFE) crystal structure for improved piezoelectric property, and increased beta phase in the crystal structure of the P(VDF-TrFE) material. The annealing temperature (Ta) range can be between 80 to 165 degrees Celcius, a temperature between the Glass transition temperature (Tg) and the melting temperature (Tm) of the P(VDF-TrFE polymer, that is, Tg<Ta<Tm. The annealing duration can be in the range from 5 minutes to 24 hours. The surface enhancement layer has gained some piezoelectric properties after the annealing process.
4. Apply topcoat: the coated stent 100 is placed in a sputtering coating chamber for ZnO topcoat for 5 minutes. The topcoat can also be TaO, TiO, ZrO and some other biocompatible dielectric polymer materials, such PTFE, Parylene, etc.
5. Poling Process: After the surface enhancement layer coating and annealing processes, the stent 100 is then polarized through the poling process to define the polarity of the device. As shown in FIG. 15, the stent 100 is loaded onto the electrode 154, and the cylindrical shaped electrode 150 is positioned over the braided device. The inner diameter of the electrode 150 is 4.5 mm. The inner diameter of the electrode 150 can be in the range between 2 mm to 20 mm depending on the diameter of the device to be poled. The electrodes 154 and 150 are connected to the power supply through the cables. The power supply is turned on and the output voltage is set to 3000 volts and kept for 30 minutes. For the finished stent 100, polarization needs to be done in a way that, under deformation, the inner surface generates a negative surface charge, while in the outer surface generates a positive charge. Therefore, during the poling process, the electrode 154 needs to be connected to the “+” output port, while the electrode 150 need to be connect to the “−” output port of the power supply. Vice versa, if the electrode 154 is connected to the “−” output port of the power supply, electrode 150 is connected with the “+” output port of the power supply, so that the resulting device will have opposite polarity, which, under deformation, the inner surface has positive charge and outer surface has negative charge.

### Example 6

A braided vascular device (such as the stent 100 shown in FIG. 10) was fabricated as follows. The stent 100 contains 64 metal wires, each with a diameter of approximately 0.0015″ (Base Materials), of which 16 were Pt (90%) alloyed with W (10%) wires, 16 were L605 Co—Cr alloy wires, 32 were Nitinol superelastic wires. The stent 100 has an outside diameter of 4 mm and a PPI along the longitudinal direction of 260, with a braid angle A of approximately 70 degrees.

Once braided, the stent 100 is cut to a length of 20 mm, and cleaned ultrasonically in ethanol solution bath. The cleaned “bare” braided device then is prepared as follows:
1. Surface activation: The surface of each stent 100 is activated in an acid solution with a composition as HF:HNO3:H2O ratio (volume %) 1:5:20 for 1 minute with ultrasonic agitation. Then the braided device is rinsed in ethanol, followed by rinsing in DI water.
2. ZnO surface enhancement layer coating process:
   2.1 Melt pure Zn material in a ceramic container at 350 degrees Celcius.
   2.2 Apply zinc ammonium chloride onto the surface of the stent 100.
   2.3 Dip the stent 100 into the molten zinc bath and hold for 5 minutes.
   2.4 Take the stent 100 out of the molten Zn bath, and quench the stent 100 in a tank filled with DI water.
   2.5 Once the temperature of the stent 100 reaches room temperature, remove the stent 100 from the quench tank, allow the stent 100 to dry, and then rinse the stent 100 in ethanol, followed by rinsing in DI water.
3. Poling Process: After the ZnO surface enhancement layer coating process, the stent 100 is then polarized through the poling process to define the polarity of the device. As shown in FIG. 15, the stent 100 is loaded onto the electrode 154, and the cylindrical shaped electrode 150 is positioned over the stent 100. The inner diameter of the electrode 150 is 4.5 mm. The inner diameter of the electrode 150 can be in the range between 2 mm to 20 mm depending on the diameter of the device to be poled. The electrodes 154 and 150 are connected to the power supply through the cables. The power supply is turned on to set the output voltage to 3000 volts and kept for 30 minutes, although the voltage can be in the range from 2000 volts to 60 Kv, and the duration can be in the range between 2 minutes to 3 hours. For the finished stent 100, polarization needs to be done in a way that, under deformation, the inner surface generates a negative surface charge, while the outer surface generates a positive charge. Therefore, during the poling process, the electrode 154 needs to be connected to the “+” output port, while the electrode 150 needs to be connect to the “−” output port of the power supply. Vice versa, if the electrode 154 is connected to the “−” output port of the power supply, the electrode 150 is connected with the “+” output port of the power supply, so that the resulting device will have opposite polarity, which, under deformation, the inner surface has positive charge and outer surface has negative charge.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method of manufacturing an implantable medical device, comprising the steps of:
providing a structural framework having a base metallic material;
coating the structural framework with a polymeric coating comprising a copolymer polymerized onto the surface of the structural framework from at least one monomer unit of (—CF2-CFH—) and at least one monomer unit of —CH2-CF2-, wherein at least one monomer has a Glass Transition Temperature (Tg) less than about 95 degrees Celsius, and wherein in homopolymer form, the at least one monomer unit of (—CF2-CFH—) in the copolymer is protonated; and
poling the coated structural framework with a desired polarity that carries either a positive charge or a negative charge on the surface of the coated structural framework.

2. The method of claim 1, wherein the copolymer is a compound having the following formula:

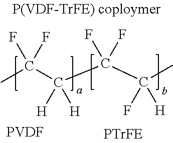

3. The method of claim 1, wherein TrFE is the polymer comprising (—CF2-CFH—).

4. The method of claim 3, wherein PVDF is the polymer comprising (—CH2-CF2-).

5. The method of claim 4, wherein the co-polymer is formed by adding PVDF into TrFE.

6. The method of claim 1, wherein the coating comprises (—CF2-CFH—) monomer in an amount from about 98% to about 2% by weight and the —CH2-CF2 monomer in an amount of from about 2% to about 98% of the weight.

7. The method of claim 1, wherein the medical device is a vascular stent, such that once implanted, under deformation, the inner surface of the vascular stents generates negative surface charge, while the outer surface of the vascular stent generates positive surface charge.

8. The method of claim 1, wherein the medical device is a vascular occlusion device, such as an embolic coil or a vascular plug, such that once implanted, under deformation, the outer surface of the vascular occlusion device generates positive surface charge, while the inner side of the vascular occlusion device generates negative surface charge.

9. The method of claim 1, further including the step of applying a top coat over the polymeric coating, wherein the top coat is a non-conductive bielectric layer.

10. The method of claim 1, wherein the base metallic material is made of nickel-titanium, Co—Cr alloy, or a combination thereof.

11. The method of claim 1, further including the step of adding a primer to either the surface of the base material before coating of the copolymer coating, or to the polymer coating.

12. A method of manufacturing an implantable medical device, comprising the steps of:
providing a structural framework having a base metallic material;
coating the structural framework with a ZnO coating; and
poling the coated structural framework with a desired polarity that carries either a positive charge or a negative charge on the surface of the coated structural framework.

13. The method of claim 12, wherein the medical device is a vascular stent, such that once implanted, under deformation, the inner surface of the vascular stents generates negative surface charge, while the outer surface of the vascular stent generates positive surface charge.

14. The method of claim 12, wherein the medical device is a vascular occlusion device, such as an embolic coil or a vascular plug, such that once implanted, under deformation, the outer surface of the vascular occlusion device generates positive surface charge, while the inner side of the vascular occlusion device generates negative surface charge

15. A method of manufacturing an implantable medical device, comprising the steps of:
providing a structural framework having a base metallic material;
partially coating the structural framework with a polymeric coating comprising a copolymer polymerized onto the surface of the structural framework from at least one monomer unit of (—CF2-CFH—) and at least one monomer unit of —CH2-CF2-, wherein at least one monomer has a Glass Transition Temperature (Tg) less than about 95 degrees Celsius, and wherein in homopolymer form, the at least one monomer unit of (—CF2-CFH—) in the copolymer is protonated;
partially coating the structural framework with a ZnO coating; and
poling the coated structural framework with a desired polarity that carries either a positive charge or a negative charge on the surface of the coated structural framework.

16. The method of claim 15, wherein the copolymer is a compound having the following formula:

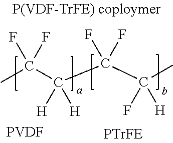

17. The method of claim 15, wherein the medical device is a vascular stent, such that once implanted, under deformation, the inner surface of the vascular stents generates negative surface charge, while the outer surface of the vascular stent generates positive surface charge.

18. The method of claim 15, wherein the medical device is a vascular occlusion device, such as an embolic coil or a vascular plug, such that once implanted, under deformation, the outer surface of the vascular occlusion device generates positive surface charge, while the inner side of the vascular occlusion device generates negative surface charge.

19. The method of claim 15, wherein TrFE is the polymer comprising (—CF2-CFH—).

20. The method of claim 19, wherein PVDF is the polymer comprising (—CH2-CF2-).
